(19) United States
(12) Patent Application Publication
(10) Pub. No.: US 2009/0053747 A1
(43) Pub. Date: Feb. 26, 2009

(54) Measurement of Haloperoxidase Activity With Chemiluminescent Detection

(76) Inventors: Phillip G. Mattingly, Third Lake, IL (US); Maciej Adamczyk, Gurnee, IL (US); Roy Jeffrey Brashear, Mundelein, IL (US)

Correspondence Address: Robert DeBerardine;D-377/AP6A-1, Abbott Laboratories, 100 Abbott Park Road, Abbott Park, IL, 60064-6008, US

(21) Appl. No.: 11/842,897

(22) Filed: Aug. 21, 2007

(51) Int. Cl. C12Q 1/28

(52) U.S. Cl. 435/28

(57) ABSTRACT

The present invention relates to assays and kits for detecting haloperoxidase activity in test samples.

### RELATED APPLICATION INFORMATION

None.

### FIELD OF THE INVENTION

The present invention relates to assays and kits for detecting or quantifying haloperoxidase activity in a test sample.

### BACKGROUND OF THE INVENTION

Haloperoxidases are a group of enzymes that are able to catalyse the halogenation of organic compounds. Specifically, haloperoxidases oxidize halides, namely, chloride (Cl⁻), bromide (Br⁻), or (I⁻) but not fluoride (Fl⁻), in the presence of a peroxide, such as hydrogen peroxide (H₂O₂), to hypohalous acid as shown below:

H₂O₂+X⁻+H⁺→H₂O+HOX (where X is the halide Cl⁻, Br⁻, or I⁻).

If a nucleophilic acceptor is present, a reaction will occur with HOX whereby a diversity of halogenated reaction products may be formed.

Haloperoxidases have been isolated from various organisms, such as, mammals, marine animals, plants, algae, lichen, fungi and bacteria. In addition to the halogenation of organic compounds, haloperoxidases have been shown to carry out sulfoxidation, epoxidation, oxidation of indoles and other specific reactions with a range of compounds. Haloperoxidases are named according to the oxidation of the most electrophilic halide that they are able to catalyze. For example, bromoperoxidases are able to oxidize iodide and bromide. Chloroperoxidases are able to oxidize chloride.

Three different groups of haloperoxidases are known. These groups are heme-thiolate containing haloperoxidases (such as chloroperoxidases from Caldariomyces fumao, canine myeloperoxidase, and a peroxidase isolated from Notomastus lobatus, myelo- and eosinophil peroxidases from human white blood cells, bovine lacto- and human thyroid peroxidases (See, Jennifer Littlechild, Current Opinion in Chemical Biology, 3:28-34 (1999) and Hofrichter, M., et al., Appl. Microbiol. Biotechnol., 71:276-288 (2006)), vanadium-containing haloperoxidases (such as vanadium bromoperoxidases from Xantheria parietina and Ascophyllum nodosum and vanadium chloroperoxidases from Caldariomyces inaequalis and Drechslera biseptate) (See, Simons, B., et al., Eur. J. Biochem., 299:566-574 (1995)), and metal-free haloperoxidases (such as, chloroperoxidases A2 from Streptomyces aureofaciens, Streptomyces lividans and Pseudomonas fluorescens (See, Jennifer Littlechild, Current Opinion in Chemical Biology, 3:28-34 (1999)).

It is known that certain types of cells generate hydrogen peroxide. Moreover, many of the same cells or types of cells are also known to secrete haloperoxidases. For example, white blood cells are known to generate hydrogen peroxide and to secrete myeloperoxidase. In the presence of hydrogen peroxide, myeloperoxidase catalyzes the oxidation of chloride to hypochlorous acid (HOCl). HOCl is a potent cytotoxin for bacteria, viruses and fungi. The generation of HOCl by white blood cells plays a key role in host defenses against invading pathogens. However, oxidant production by phagocytic white cells is also potentially deleterious and is believed to represent an important pathway for tissue damage in disorders ranging from arthritis to ischemia reperfusion injury to cancer.

Oxidative injury is believed to be of central importance in promoting atherosclerotic heart disease. One risk factor in atherosclerosis is elevated levels of low density lipoprotein (“LDL”). In vitro, LDL fails to exert effects that would promote heart disease in vivo. However, oxidation of LDL, renders the lipoprotein atherogenic. Many lines of evidence indicate that the oxidation of LDL is of central importance in the promotion of heart disease. Oxidized LDL has been isolated from atherosclerotic lesions and antioxidants have been found to retard atherosclerosis in animals.

Elevated levels of haloperoxidases, such as myeloperoxidase (MPO), in subjects with cardiovascular disease, have been associated with arterial inflammation. A number of studies have linked arterial inflammation with an increased risk of cardiovascular events. Additionally, recent studies have shown that serum myeloperoxidase levels are associated with the future risk of coronary artery disease in apparently healthy individuals (See, Marijn C. Meuwese et al., Journal of the American College of Cardiology, 50(2):159-165 (2007)). The measurement in test samples such as blood of the levels of haloperoxidases such as myeloperoxidase are used to predict whether or not an individual is at risk of developing cardiovascular disease, such as coronary heart disease.

Therefore, there is a need in the art for new methods of detecting or determining haloperoxidase activity in a test sample.

### SUMMARY OF THE PRESENT INVENTION

In one embodiment, the present invention relates to a method of detecting haloperoxidase activity in a test sample. The method comprises the steps of:

a) adding an acridinium-9-carboxamide to a test sample;

b) generating in or providing to the test sample a source of hydrogen peroxide before or after the addition of an acridinium-9-carboxamide;

c) adding a basic solution to the test sample to generate a light signal; and

d) measuring the light generated to detect the haloperoxidase.

The test sample used in the above-described method can be whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen. The haloperoxidase that can be used in the above-described method can be selected from the group consisting of: myeloperoxidase, thyroperoxidase, eosinoperoxidase, eosinophil peroxidase and lactoperoxidase.

In the above-described method, the source of the hydrogen peroxide can be provided by adding a buffer or a solution containing hydrogen peroxide. Alternatively, the hydrogen peroxide is generated by adding a hydrogen peroxide generating enzyme to the test sample.

In the above-described method, the acridinium-9-carboxamide has a structure according to Formula I:

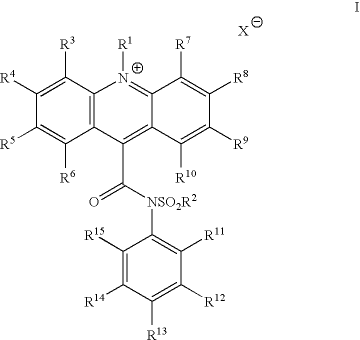

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Additionally, the above method further optionally comprises quantifying the activity of haloperoxidase in the test sample by relating the amount of light generated in the test sample by comparison to a standard curve for said haloperoxidase. Also, optionally, the standard curve can be generated from solutions of a haloperoxidase of a known concentration.

In another embodiment, the present invention relates to a kit for use in detecting haloperoxidase activity in a test sample. The kit can comprise:
a. at least one acridinium-9-carboxamide;
b. at least one basic solution;
c. a source of hydrogen peroxide; and
d. instructions for detecting haloperoxidase activity in a test sample.

The at least one acridinium-9-carboxamide in the above kit can have a structure according to formula I:

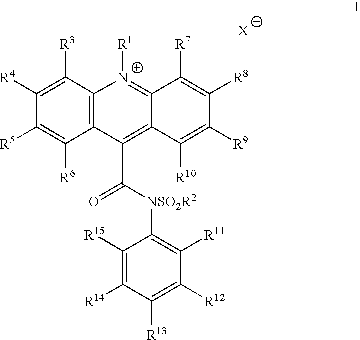

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

The source of hydrogen peroxide in the above-described kit can be a buffer or a solution containing hydrogen peroxide.

In another embodiment, the present invention relates to another kit for use in detecting haloperoxidase activity in a test sample. The kit can comprise:
a. at least one acridinium-9-carboxamide;
b. at least one basic solution;
c. a means of generating hydrogen peroxide in situ in the test sample; and
d. instructions for detecting haloperoxidase activity in a test sample.

The at least one acridinium-9-carboxamide in the above kit can have a structure according to formula I:

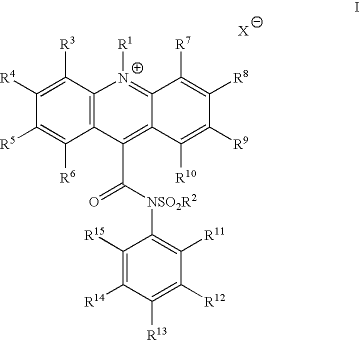

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

The above-described kit can further comprise instructions for generating hydrogen peroxide in situ in the test sample.

Additionally, the means for generating hydrogen peroxide in situ in the test sample contained in the kit can be at least one hydrogen peroxide generating enzyme. The at least one hydrogen peroxide generating enzyme can be selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate)oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, AND(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

### BRIEF DESCRIPTION OF THE FIGURES

FIG. 1 is a plot showing myeloperoxidase (MPO) dose response at 150 μM hydrogen peroxide tested pursuant to Example 1.

FIG. 2 is a plot showing MPO dose response at 75 μM hydrogen peroxide tested pursuant to Example 1.

FIG. 3 is a plot showing MPO dose response at 50 μM hydrogen peroxide tested pursuant to Example 1.

FIG. 4 is a plot showing MPO dose response at 30 μM hydrogen peroxide tested pursuant to Example 1.

FIG. 5 is a plot showing MPO dose response at 20 μM hydrogen peroxide tested pursuant to Example 1.

FIG. 6 is a plot showing MPO dose response at 10 μM hydrogen peroxide tested pursuant to Example 1.

FIG. 7 is a plot showing MPO dose response at 5 μM hydrogen peroxide tested pursuant to Example 1.

FIG. 8 is a plot showing MPO dose response at 0 μM hydrogen peroxide tested pursuant to Example 1.

FIG. 9 is a graph the effect of sodium hypochlorite (100 μM) and methionine (1 mM) on the generation of hydrogen peroxide from choline and choline oxidase tested pursuant to Example 2. ♦ shows the standards, ▪ shows the standards plus 100 μM sodium hypochlorite, and ▴ shows the standards plus 100 μM sodium hypochlorite and 1 mM methionine.

FIG. 10 is a plot showing the dose response of MPO with the generation of hydrogen peroxide from 150 μM choline and choline oxidase tested pursuant to Example 3.

FIG. 11 is a plot showing the dose response of MPO with the generation of hydrogen peroxide from 75 μM choline and choline oxidase tested pursuant to Example 3.

FIG. 12 is a plot showing the dose response of MPO with the generation of hydrogen peroxide from 50 μM choline and choline oxidase tested pursuant to Example 3.

FIG. 13 is a plot showing the dose response of MPO with the generation of hydrogen peroxide from 30 μM choline and choline oxidase tested pursuant to Example 3.

FIG. 14 is a plot showing the dose response of MPO with the generation of hydrogen peroxide from 20 μM choline and choline oxidase tested pursuant to Example 3.

FIG. 15 is a plot showing the dose response of MPO with the generation of hydrogen peroxide from 10 μM choline and choline oxidase tested pursuant to Example 3.

FIG. 16 is a plot showing the dose response of MPO with the generation of hydrogen peroxide from 5 μM choline and choline oxidase tested pursuant to Example 3.

FIG. 17 is a plot showing the dose response of MPO with the generation of hydrogen peroxide from 0 μM choline and choline oxidase tested pursuant to Example 3.

FIG. 18 is a plot showing the dose response of MPO with the generation of hydrogen peroxide from 150 μM choline and choline oxidase at 37° C. tested pursuant to Example 4.

FIG. 19 is a plot showing the dose response of MPO with the generation of hydrogen peroxide from 75 μM choline and choline oxidase at 37° C. tested pursuant to Example 4.

FIG. 20 is a plot showing the dose response of MPO with the generation of hydrogen peroxide from 50 μM choline and choline oxidase, at 37° C. tested pursuant to Example 4.

FIG. 21 is a plot showing the dose response of MPO with the generation of hydrogen peroxide from 30 μM choline and choline oxidase, at 37° C. tested pursuant to Example 4.

FIG. 22 is a plot showing the dose response of MPO with the generation of hydrogen peroxide from 20 μM choline and choline oxidase, at 37° C. tested pursuant to Example 4.

FIG. 23 is a plot showing the dose response of MPO with the generation of hydrogen peroxide from 10 μM choline and choline oxidase, at 37° C. tested pursuant to Example 4.

FIG. 24 is a plot showing the dose response of MPO with the generation of hydrogen peroxide from 5 μM choline and choline oxidase, at 37° C. tested pursuant to Example 4.

FIG. 25 is a plot showing the dose response of MPO with the generation of hydrogen peroxide from 0 μM choline and choline oxidase, at 37° C. tested pursuant to Example 4.

FIG. 26 is a plot showing the dose response of MPO with the in situ generation of hydrogen peroxide from 150 μM choline and choline oxidase, 30 minutes, at 37° C. tested pursuant to Example 5.

FIG. 27 is a plot showing the dose response of MPO with the in situ generation of hydrogen peroxide from 75 μM choline and choline oxidase, 30 minutes, at 37° C. tested pursuant to Example 5.

FIG. 28 is a plot showing the dose response of MPO with the in situ generation of hydrogen peroxide from 50 μM choline and choline oxidase, 30 minutes, at 37° C. tested pursuant to Example 5.

FIG. 29 is a plot showing the dose response of MPO with the in situ generation of hydrogen peroxide from 30 μM choline and choline oxidase, 30 minutes, at 37° C. tested pursuant to Example 5.

FIG. 30 is a plot showing the dose response of MPO with the in situ generation of hydrogen peroxide from 20 μM choline and choline oxidase, 30 minutes, at 37° C. tested pursuant to Example 5.

FIG. 31 is a plot showing the dose response of MPO with the in situ generation of hydrogen peroxide from 10 μM choline and choline oxidase, 30 minutes, at 37° C. tested pursuant to Example 5.

FIG. 32 is a plot showing the dose response of MPO with the in situ generation of hydrogen peroxide from 5 μM choline and choline oxidase, 30 minutes, at 37° C. tested pursuant to Example 5.

FIG. 33 is a plot showing the dose response of myeloperoxidase with the in situ generation of hydrogen peroxide from 0 μM choline and choline oxidase, 30 minutes, at 37° C. tested pursuant to Example 5.

FIG. 34 shows the structure of 9-[[(3-Carboxypropyl)[(4-methylphenyl)sulfonyl]amino]-carbonyl]-10-(3-sulfopropyl)acridinium inner salt.

### DETAILED DESCRIPTION OF THE PRESENT INVENTION

The present invention provides assays and kits for detecting haloperoxidase activity in a test sample.

A. Definitions

As used herein, the term “acyl” refers to a —C(O)Rₐ group where Rₐ is hydrogen, alkyl, cycloalkyl, cycloalkylalkyl, phenyl or phenylalkyl. Representative examples of acyl include, but are not limited to, formyl, acetyl, cylcohexylcarbonyl, cyclohexylmethylcarbonyl, benzoyl, benzylcarbonyl and the like.

As used herein, the term “alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As used herein, the term “alkyl radical” means any of a series of univalent groups of the general formula CₙH₂ₙ₊₁ derived from straight or branched chain hydrocarbons.

As used herein, the term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, the term “amido” refers to an amino group attached to the parent molecular moiety through a carbonyl group (wherein the term “carbonyl group” refers to a —C(O)— group).

As used herein, the term “amino” means —NR_{b}R_{c}, wherein R_{b} and R_{c} are independently selected from the group consisting of hydrogen, alkyl and alkylcarbonyl.

As used herein, the term “anion” refers to an anion of an inorganic or organic acid, such as, but not limited to, hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, aspartic acid, phosphate, trifluoromethansulfonic acid, trifluoroacetic acid and fluorosulfonic acid and any combinations thereof.

As used herein, the term “aralkyl” means an aryl group appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

As used herein, the term “aryl” means a phenyl group, or a bicyclic or tricyclic fused ring system wherein one or more of the fused rings is a phenyl group. Bicyclic fused ring systems are exemplified by a phenyl group fused to a cycloalkenyl group, a cycloalkyl group, or another phenyl group. Tricyclic fused ring systems are exemplified by a bicyclic fused ring system fused to a cycloalkenyl group, a cycloalkyl group, as defined herein or another phenyl group. Representative examples of aryl include, but are not limited to, anthracenyl, azulenyl, fluorenyl, indanyl, indenyl, naphthyl, phenyl, and tetrahydronaphthyl. The aryl groups of the present invention can be optionally substituted with one-, two, three, four, or five substituents independently selected from the group consisting of alkoxy, alkyl, carboxyl, halo, and hydroxyl.

As used herein, the term “carboxy” or “carboxyl” refers to —CO₂H or —CO₂⁻.

As used herein, the term “carboxyalkyl” refers to a —(CH₂)ₙCO₂H or —(CH₂)ₙCO₂⁻ group where n is from 1 to 10.

As used herein, the term “cyano” means a —CN group.

As used herein, the term “cycloalkenyl” refers to a non-aromatic cyclic or bicyclic ring system having from three to ten carbon atoms and one to three rings, wherein each five-membered ring has one double bond, each six-membered ring has one or two double bonds, each seven- and eight-membered ring has one to three double bonds, and each nine-to ten-membered ring has one to four double bonds. Representative examples of cycloalkenyl groups include cyclohexenyl, octahydronaphthalenyl, norbornylenyl, and the like. The cycloalkenyl groups can be optionally substituted with one, two, three, four, or five substituents independently selected from the group consisting of alkoxy, alkyl, carboxyl, halo, and hydroxyl.

As used herein, the term “cycloalkyl” refers to a saturated monocyclic, bicyclic, or tricyclic hydrocarbon ring system having three to twelve carbon atoms. Representative examples of cycloalkyl groups include cyclopropyl, cyclopentyl, bicyclo[3.1.1]heptyl, adamantyl, and the like. The cycloalkyl groups of the present invention can be optionally substituted with one, two, three, four, or five substituents independently selected from the group consisting of alkoxy, alkyl, carboxyl, halo, and hydroxyl.

As used herein, the term “cycloalkylalkyl” means a —R_{d}Rₑ group where R_{d} is an alkylene group and Rₑ is cycloalkyl group. A representative example of a cycloalkylalkyl group is cyclohexylmethyl and the like.

As used herein, the term “halogen” means a —Cl, —Br, —I or —F; the term “halide” means a binary compound, of which one part is a halogen atom and the other part is an element or radical that is less electronegative than the halogen, e.g., an alkyl radical.

As used herein, the term “hydroxyl” means an —OH group.

As used herein, the term “nitro” means a —NO₂ group.

As used herein, the term “oxoalkyl” refers to —(CH₂)ₙC(O)Rₐ, where Rₐ is hydrogen, alkyl, cycloalkyl, cycloalkylalkyl, phenyl or phenylalkyl and where n is from 1 to 10.

As used herein, the term “phenylalkyl” means an alkyl group which is substituted by a phenyl group.

As used herein, the term “sulfo” means a —SO₃H group.

As used herein, the term “sulfoalkyl” refers to a —(CH₂)ₙSO₃H or —(CH₂)ₙSO₃⁻ group where n is from 1 to 10.

As used herein, the term “test sample” generally refers to a biological material being tested for and/or suspected of containing an analyte of interest, such as a haloperoxidase. The test sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

As used in this specification and the appended claims, the singular forms “a”, “an”, and “the” include plural references unless the context clearly dictates otherwise. Thus, for example, a reference to “the method” includes one or more methods, and/or steps of the type described herein and/or which will become apparent to those persons skilled in the art upon reading this disclosure and so forth.

B. Assay for Detecting or Quantifying Haloperoxidase Activity

In general, the present invention relates to an assay for detecting or quantifying haloperoxidase activity in a test sample. As used herein, the “haloperoxidase activity” refers to the turnover or consumption of a substrate based on a quantifiable amount (e.g., mass) of a haloperoxidase. In other words, haloperoxidase activity refers to the amount of haloperoxidase needed to convert or change a substrate into the requisite product in a given time.

1. Test Sample

The assay or method of the present invention involves obtaining a test sample from a subject. A subject from which a test sample can be obtained is any vertebrate. Preferably, the vertebrate is a mammal. Examples of mammals include, but are not limited to, dogs, cats, rabbits, mice, rats, goats, sheep, cows, pigs, horses, non-human primates and humans. The test sample can be obtained from the subject using routine techniques known to those skilled in the art. Preferably, the test sample contains one or more haloperoxidases, such as, but not limited to, myeloperoxidase, thyroperoxidase (TPO), eosinoperoxidase (EPO, eosinophil peroxidase), lactoperoxidase or any combinations thereof. Optionally, the test sample contains cells which produce or secrete one or more haloperoxidases, such as, but not limited to, myeloperoxidase, thyroperoxidase (TPO), eosinoperoxidase (EPO, eosinophil peroxidase), lactoperoxidase or any combinations thereof.

2. Acridinium-9-carboxamide

After the test sample is obtained from a subject, at least one acridinium carboxamide is added to the test sample. Preferably, the acridinium carboxamide is an acridinium-9-carboxamide, including optionally an acridinium-9-carboamide having a structure according to formula I shown below:

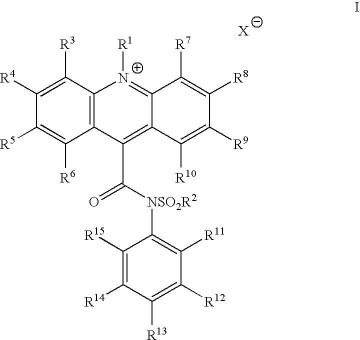

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and further wherein any of the alkyl, alkenyl, alkynyl, aryl or aralkyl may contain one or more heteroatoms; and

optionally, if present, X^{⊖} is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524, and 5,783,699 (each incorporated herein by reference in their entireties for their teachings regarding same).

3. Hydrogen Peroxide Source

In one embodiment of the present invention, hydrogen peroxide is generated in situ in the test sample or provided or supplied to the test sample before the addition of the above-described acridinium-9-carboxamide. In a second embodiment of the present invention, the hydrogen peroxide is generated in situ in the test ample or provided or supplied to the test sample simultaneously with the above-described acridinium-9-carboxamide. In a third embodiment, hydrogen peroxide is generated in situ or provided or supplied to the test sample after the above-described acridinium-9-carboxamide is added to the test sample.

As mentioned above, hydrogen peroxide can be generated in situ in the test sample. Hydrogen peroxide can be generated in situ in a number of ways. For example, a number of enzymes are known in the art that are capable of generating hydrogen peroxide (which is also referred to herein as a hydrogen peroxide generating enzyme). Such enzymes are listed below in Table 1.

TABLE 1

| | IUBMB | |
|---|---|---|
| | ENZYME | |
| | NOMEN- | PREFERRED |
| ACCEPTED COMMON NAME | CLATURE | SUBSTRATE |
| (R)-6-hydroxynicotine oxidase | EC 1.5.3.6 | (R)-6-hydroxynicotine |
| (S)-2-hydroxy acid oxidase | EC 1.1.3.15 | S)-2-hydroxy acid |
| (S)-6-hydroxynicotine oxidase | EC 1.5.3.5 | (S)-6-hydroxynicotine |
| 3-aci-nitropropanoate oxidase | EC 1.7.3.5 | 3-aci-nitropropanoate |
| 3-hydroxyanthranilate oxidase | EC 1.10.3.5 | 3-hydroxyanthranilate |
| 4-hydroxymandelate oxidase | EC 1.1.3.19 | (S)-2-hydroxy-2-(4- |
| | | hydroxyphenyl)acetate |
| 6-hydroxynicotinate | EC 1.17.3.3 | 6-hydroxynicotinate |
| dehydrogenase | | |
| Abscisic-aldehyde oxidase | EC 1.2.3.14 | abscisic aldehyde |
| acyl-CoA oxidase | EC 1.3.3.6 | acyl-CoA |
| Alcohol oxidase | EC 1.1.3.13 | a primary alcohol |
| aldehyde oxidase | EC 1.2.3.1 | an aldehyde |
| amine oxidase | | |
| amine oxidase | EC 1.4.3.6 | primary monoamines, |
| (copper-containing) | | diamines and histamine |
| amine oxidase | EC 1.4.3.4 | a primary amine |
| (flavin-containing) | | |
| aryl-alcohol oxidase | EC 1.1.3.7 | an aromatic primary |
| | | alcohol |
| | | (2-naphthyl)methanol |
| | | 3-methoxybenzyl alcohol |
| aryl-aldehyde oxidase | EC 1.2.3.9 | an aromatic aldehyde |
| catechol oxidase | EC 1.1.3.14 | Catechol |
| cholesterol oxidase | EC 1.1.3.6 | Cholesterol |
| choline oxidase | EC 1.1.3.17 | Choline |
| columbamine oxidase | EC 1.21.3.2 | Columbamine |
| cyclohexylamine oxidase | EC 1.4.3.12 | Cyclohexylamine |
| cytochrome c oxidase | EC 1.9.3.1 | |
| D-amino-acid oxidase | EC 1.4.3.3 | a D-amino acid |
| D-arabinono-1,4-lactone oxidase | EC 1.1.3.37 | D-arabinono-1,4-lactone |
| D-arabinono-1,4-lactone oxidase | EC 1.1.3.37 | D-arabinono-1,4-lactone |
| D-aspartate oxidase | EC 1.4.3.1 | D-aspartate |
| D-glutamate oxidase | EC 1.4.3.7 | D-glutamate |
| D-glutamate(D-aspartate) | EC 1.4.3.15 | D-glutamate |
| oxidase | | |
| dihydrobenzophenanthridine | EC 1.5.3.12 | dihydrosanguinarine |
| oxidase | | |
| dihydroorotate oxidase | EC 1.3.3.1 | (S)-dihydroorotate |
| dihydrouracil oxidase | EC 1.3.3.7 | 5,6-dihydrouracil |
| dimethylglycine oxidase | EC 1.5.3.10 | N,N-dimethylglycine |
| D-mannitol oxidase | EC 1.1.3.40 | Mannitol |
| ecdysone oxidase | EC 1.1.3.16 | Ecdysone |
| ethanolamine oxidase | EC 1.4.3.8 | Ethanolamine |
| galactose oxidase | EC 1.1.3.9 | D-galactose |
| glucose oxidase | EC 1.1.3.4 | β-D-glucose |
| glutathione oxidase | EC 1.8.3.3 | Glutathione |
| glycerol-3-phosphate oxidase | EC 1.1.3.21 | sn-glycerol 3-phosphate |
| glycine oxidase | EC 1.4.3.19 | Glycine |
| glyoxylate oxidase | EC 1.2.3.5 | Glyoxylate |
| hexose oxidase | EC 1.1.3.5 | D-glucose, |
| | | D-galactose |
| | | D-mannose |
| | | maltose |
| | | lactose |
| | | cellobiose |
| hydroxyphytanate oxidase | EC 1.1.3.27 | L-2-hydroxyphytanate |
| indole-3-acetaldehyde oxidase | EC 1.2.3.7 | (indol-3-yl)acetaldehyde |
| lactic acid oxidase | | Lactic acid |
| L-amino-acid oxidase | EC 1.4.3.2 | an L-amino acid |
| L-aspartate oxidase | EC 1.4.3.16 | L-aspartate |
| L-galactonolactone oxidase | EC 1.3.3.12 | L-galactono-1,4-lactone |
| L-glutamate oxidase | EC 1.4.3.11 | L-glutamate |
| L-gulonolactone oxidase | EC 1.1.3.8 | L-gulono-1,4-lactone |
| L-lysine 6-oxidase | EC 1.4.3.20 | L-lysine |
| L-lysine oxidase | EC 1.4.3.14 | L-lysine |
| long-chain-alcohol oxidase | EC 1.1.3.20 | A long-chain-alcohol |
| L-pipecolate oxidase | EC 1.5.3.7 | L-pipecolate |
| L-sorbose oxidase | EC 1.1.3.11 | L-sorbose |
| malate oxidase | EC 1.1.3.3 | (S)-malate |
| methanethiol oxidase | EC 1.8.3.4 | Methanethiol |
| monoamino acid oxidase | | |
| N⁶-methyl-lysine oxidase | EC 1.5.3.4 | 6-N-methyl-L-lysine |
| N-acylhexosamine oxidase | EC 1.1.3.29 | N-acetyl-D-glucosamine |
| | | N-glycolylglucosamine |
| | | N-acetylgalactosamine |
| | | N-acetylmannosamine. |
| NAD(P)H oxidase | EC 1.6.3.1 | NAD(P)H |
| nitroalkane oxidase | EC 1.7.3.1 | a nitroalkane |
| N-methyl-L-amino-acid oxidase | EC 1.5.3.2 | an N-methyl-L-amino |
| | | acid |
| nucleoside oxidase | EC 1.1.3.39 | Adenosine |
| oxalate oxidase | EC 1.2.3.4 | Oxalate |
| polyamine oxidase | EC 1.5.3.11 | 1-N-acetylspermine |
| polyphenol oxidase | EC 1.14.18.1 | |
| polyvinyl-alcohol oxidase | EC 1.1.3.30 | polyvinyl alcohol |
| prenylcysteine oxidase | EC 1.8.3.5 | an S-prenyl-L-cysteine |
| protein-lysine 6-oxidase | EC 1.4.3.13 | peptidyl-L-lysyl-peptide |
| putrescine oxidase | EC 1.4.3.10 | butane-1,4-diamine |
| pyranose oxidase | EC 1.1.3.10 | D-glucose |
| | | D-xylose |
| | | L-sorbose |
| | | D-glucono-1,5-lactone |
| pyridoxal 5′-phosphate synthase | EC 1.4.3.5 | pyridoxamine 5′- |
| | | phosphate |
| pyridoxine 4-oxidase | EC 1.1.3.12 | Pyridoxine |
| pyrroloquinoline-quinone | EC 1.3.3.11 | 6-(2-amino-2- |
| synthase | | carboxyethyl)-7,8-dioxo- |
| | | 1,2,3,4,5,6,7,8- |
| | | octahydroquinoline-2,4- |
| | | dicarboxylate |
| pyruvate oxidase | EC 1.2.3.3 | Pyruvate |
| pyruvate oxidase | EC 1.2.3.6 | Pyruvate |
| (CoA-acetylating) | | |
| reticuline oxidase | EC 1.21.3.3 | Reticuline |
| retinal oxidase | EC 1.2.3.11 | Retinal |
| rifamycin-B oxidase | EC 1.10.3.6 | rifamycin-B |
| sarcosine oxidase | EC 1.5.3.1 | Sarcosine |
| secondary-alcohol oxidase | EC 1.1.3.18 | a secondary alcohol |
| sulfite oxidase | EC 1.8.3.1 | Sulfite |
| superoxide dismutase | EC 1.15.1.1 | Superoxide |
| superoxide reductase | EC 1.15.1.2 | Superoxide |
| tetrahydroberberine oxidase | EC 1.3.3.8 | (S)-tetrahydroberberine |
| thiamine oxidase | EC 1.1.3.23 | Thiamine |
| tryptophan α,β-oxidase | EC 1.3.3.10 | L-tryptophan |
| urate oxidase (uricase, uric acid | EC 1.7.3.3 | uric acid |
| oxidase) | | |
| vanillyl-alcohol oxidase | EC 1.1.3.38 | vanillyl alcohol |
| xanthine oxidase | EC 1.17.3.2 | Xanthine |
| xylitol oxidase | EC 1.1.3.41 | Xylitol |

One or more of the above-described enzymes can be added to the test sample in an amount sufficient to allow for the generation of hydrogen peroxide in situ in the test sample. The amount of one or more of the above enzymes to be added to the test sample can be readily determined by one skilled in the art.

Hydrogen peroxide can also be generated electrochemically in situ as shown in Agladze, G. R.; Tsurtsumia, G. S.; Jung, B. I.; Kim, J. S.; Gorelishvili, G. J. Applied Electrochem., 37, 375-383 (2007); Qiang, Z.; Chang, J.-H.; Huang, C.-P. Water Research, 36, 85-94 (2002), for example. Hydrogen peroxide can also be generated photochemically in situ, e.g. Draper, W. M.; Crosby, D. G. Archives of Environmental Contamination and Toxicology, 12, 121-126 (1983).

Alternatively, a source of hydrogen peroxide can be supplied to or provided in the test sample. For example, the source of the hydrogen peroxide can be one or more buffers or other solutions that are known to contain hydrogen peroxide. Such buffers or other solutions are simply added to the test sample. Alternatively, another source of hydrogen peroxide can simply be a solution containing hydrogen peroxide.

The amount of hydrogen peroxide generated in situ in the test sample or provided in or supplied to the test sample can be readily determined by one skilled in the art depending on the activity of the specific haloperoxidase to be detected or determined pursuant to the assay of the present invention. For example, if the haloperoxidase activity to be detected or determined is the activity of myeloperoxidase, then the amount of hydrogen peroxide that can be generated in situ or provided in or supplied to the test sample is from about 0.0001 micromolar to about 200 micromolar.

As demonstrated by the above, the timing and order in which the acridinium-9-carboxamide and the hydrogen peroxide provided in or supplied to or generated in situ in the test sample is not critical provided that they are added, provided, supplied or generated in situ prior to the addition of at least one basic solution, which will be discussed in more detail below.

After the addition of the acridinium-9-carboxamide having the formula of formula I and the hydrogen peroxide to the test sample, at least one basic solution is added to the test sample in order to generate a detectable signal, namely, a chemiluminescent signal. The basic solution is the same basic solution discussed previously herein, namely, a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the test sample depends on the concentration of the basic solution used in the assay. Based on the concentration of the basic solution used, one skilled in the art could easily determine the amount of basic solution to be used in the method. Chemiluminescent signals generated can be detected using routine techniques known to those skilled in the art.

4. Signal Generation

Thus, in the assay of the present invention, the chemiluminescent signal generated after the addition of a basic solution, indicates the activity of a haloperoxidase. The activity of the haloperoxidase in the test sample can be quantified based on the intensity of the signal generated. Specifically, the activity of haloperoxidase contained in a test sample is inversely proportional to the intensity of the signal generated. For example, in some instances, a high signal intensity may be generated by the lowest concentration of haloperoxidase in the test sample (in this instance, the activity of haloperoxidase in the test sample is inversely proportional to the amount of signal generated). Specifically, the activity of haloperoxidase present can be quantified based on comparing the amount of light generated to a standard curve for the haloperoxidase or by comparison to a reference standard. The standard curve can be generated using serial dilutions or solutions of the haloperoxidase of known concentration, by mass spectroscopy, gravimetrically and by other techniques known in the art.

C. Kit for Detecting or Quantifying Haloperoxidase Activity

In another embodiment, the present invention relates to a kit for determining or detecting haloperoxidase activity in a test sample. In one aspect, the kit can contain at least one acridinium-9-carboxamide having the structure according to Formula I:

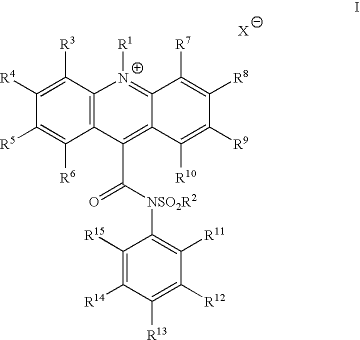

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and further wherein any of the alkyl, alkenyl, alkynyl, aryl or aralkyl may contain one or more heteroatoms; and

optionally, if present, X^{⊖} is an anion.

Additionally, the kit can also contain a source of hydrogen peroxide, such as one or more buffers or one or more solutions containing hydrogen peroxide. Furthermore, the kit can contain also at least one basic solution.

In yet another embodiment, the present invention relates to another kit for determining or detecting haloperoxidase activity in a test sample. In one aspect, the kit can contain at least one acridinium-9-carboxamide having a structure according to formula I:

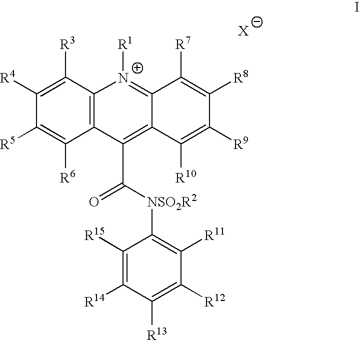

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and further wherein any of the alkyl, alkenyl, alkynyl, aryl or aralkyl may contain one or more heteroatoms; and

optionally, if present, X^{⊖} is an anion.

Additionally, the kit can also contain one basic solution. Moreover, the kit can also contain a means of generating hydrogen peroxide in situ in the test sample. A means for generating hydrogen peroxide in situ in the test sample can include adding at least one hydrogen peroxide generating enzyme. A hydrogen peroxide generating enzyme that can be used can be selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

Also, the kit can also contain one or more instructions for detecting haloperoxidase activity in a test sample. The kit can also contain instructions for generating a standard curve for the purposes of quantifying the activity of the haloperoxidase activity in the test sample or a reference standard for purposes of quantifying the activity of haloperoxidase activity in the test sample. Moreover, the kit can also contain instructions for generating hydrogen peroxide in situ in the test sample. Such instructions optionally can be in printed form or on CD, DVD, or other format of recorded media.

By way of example, and not of limitation, examples of the present invention shall now be provided.

### EXAMPLE 1

### Dose Response for Detection of Myeloperoxidase

### Standard Solutions of Hydrogen Peroxide.

Hydrogen Peroxide (30% aq, ACS grade, JTBaker #2186-01) was diluted in reagent grade water to give solutions of 150, 75, 50, 30, 20, 10, 5 and 0 μM.

### Standard Solution of Myeloperoxidase.

Myeloperoxidase from human leukocytes (Sigma #M6908) was diluted in phosphate buffered saline (PBS, pH 7.2) containing methionine (1 mM) to give solutions of 2900, 1450, 725, 362.50, 181.25, 90.63, 45.31, 22.66, 11.33, and 0.00 ng/mL.

### Chemiluminescent Detection Reagent.

9-[[(3-Carboxypropyl)[(4-methylphenyl)sulfonyl]amino]-carbonyl]-10-(3-sulfopropyl)acridinium inner salt was dissolved in reagent grade water containing sodium cholate (0.1% wt/vol) to give a concentration of 250 nM.

### Assay Protocol.

The standard hydrogen peroxide solutions (4 μL) and the standard myeloperoxidase solutions (20 μL) were arrayed in triplicate in a 96-well microplate. The plate was incubated in a microplate luminometer (Mithras LB-940, BERTHOLD TECHNOLOGIES U.S.A. LLC, Oak Ridge, Tenn.) at 37° C. for 30 minutes. Well by well, the chemiluminescent detection reagent (40 μL) and aqueous sodium hydroxide (0.25 N, 100 μL) were sequentially added and the chemiluminescent signal was recorded for 2 s.

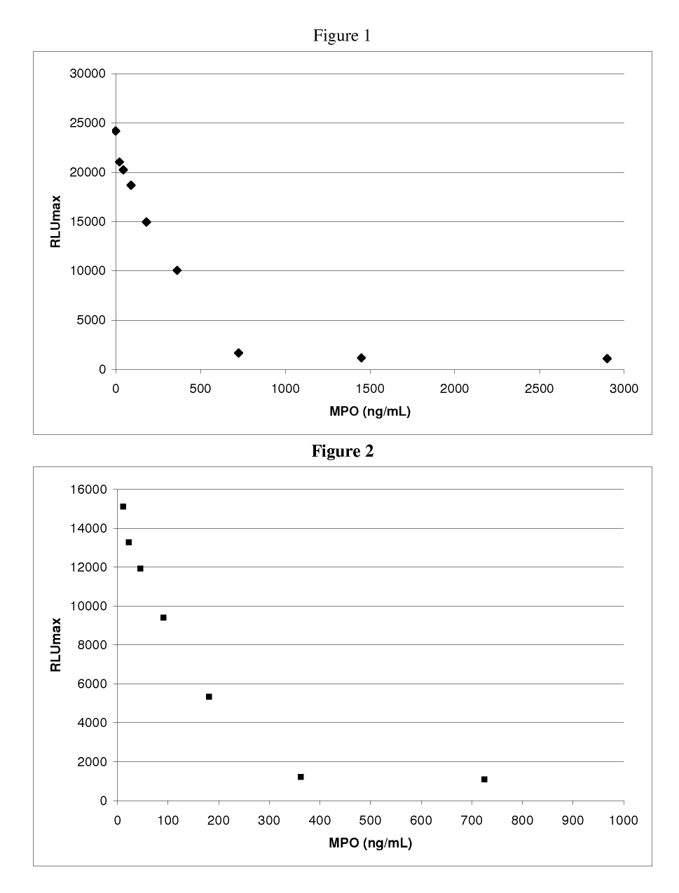

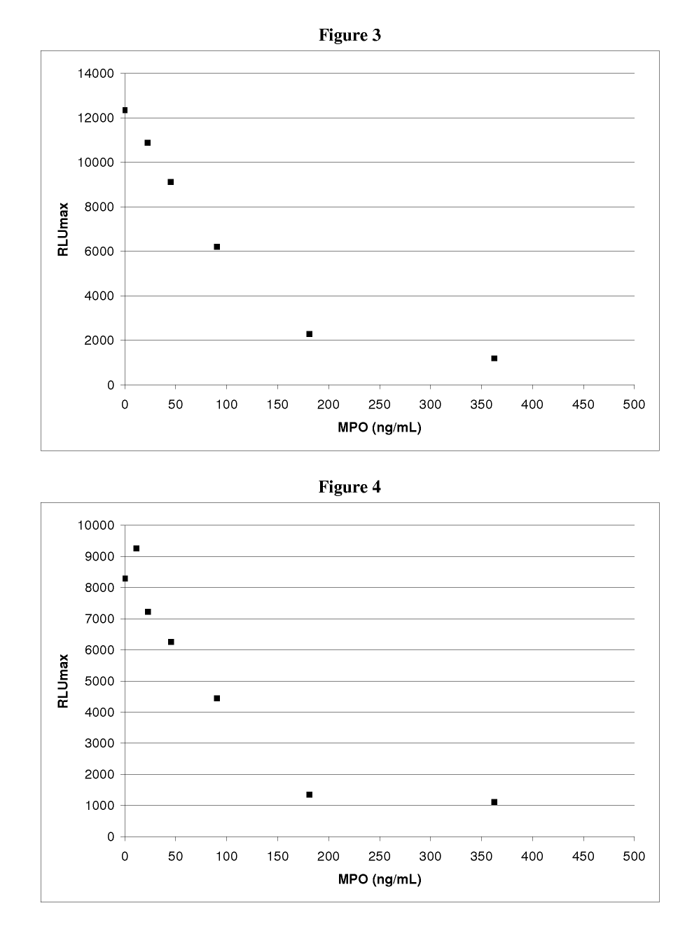

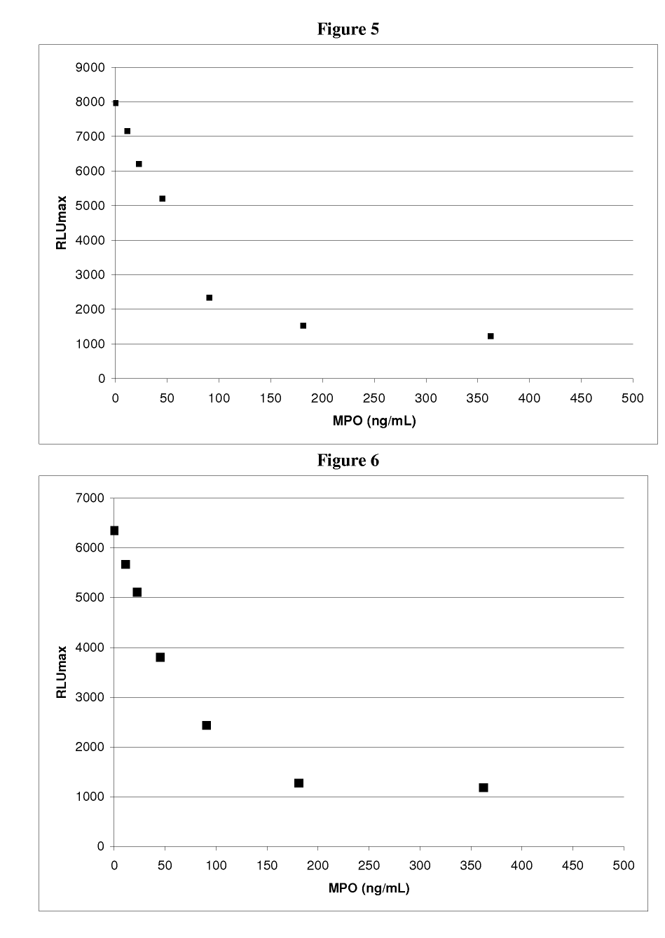

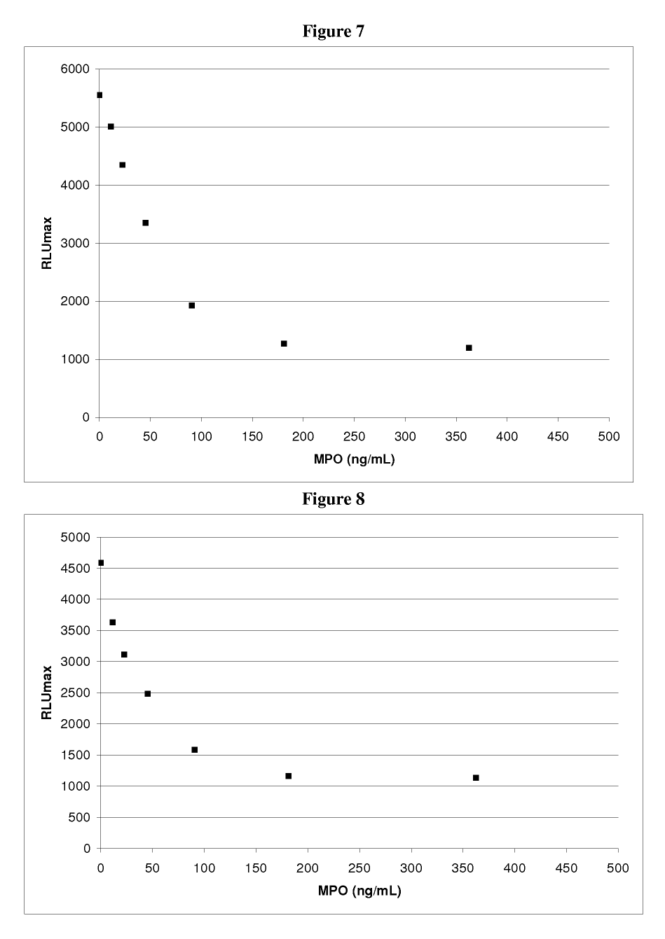

The resulting dose response (chemiluminescent signal/myeloperoxidase concentration) at each of the hydrogen peroxide concentrations tested is shown in FIGS. 1 to 8.

### EXAMPLE 2

### Effect of Hypochlorite and Methionine on Choline/Choline Oxidase Chemiluminescent Assay

### Sodium Hypochlorite Solution.

Sodium Hypochlorite 5.25 wt % (commercial bleach) was diluted in phosphate buffered saline (PBS, 100 mM phosphate, 150 mM NaCl, pH 7.2) to give a 100 μM solution.

### Methionine Solution.

Methionine was dissolved in phosphate buffered saline (PBS, 100 mM phosphate, 150 mM NaCl, pH 7.2) to give a 1 mM solution.

### Choline Standard Solutions.

Choline standards (0, 5, 10, 20, 30, 50, 75, and 150 μM in phosphate buffer, 0.2 M, pH 8) were prepared as reported (Adamczyk M, Brashear R J, Mattingly P G, Tsatsos P H. Homogeneous chemiluminescent assays for free choline in human plasma and whole blood. Anal Chim Acta. 579(1):61-7 (2006)).

### Choline Oxidase Reagent.

Choline oxidase (10 U/mL in phosphate buffer, 0.2 M, pH 8; 0.1% sodium cholate) were prepared as reported (Adamczyk M, Brashear R J, Mattingly P G, Tsatsos P H. Homogeneous chemiluminescent assays for free choline in human plasma and whole blood. Anal Chim Acta. 579(1):61-7 (2006)).

### Chemiluminescent Detection Reagent.

9-[[(3-Carboxypropyl)[(4-methylphenyl)sulfonyl]amino]-carbonyl]-10-(3-sulfopropyl)acridinium inner salt was dissolved in reagent grade water containing sodium cholate (0.1% wt/vol) to give a concentration of 4 μM.

### Assay Protocol.

The choline standard solutions (4 μL) and either PBS (8 μL), 1:1 PBS/sodium hypochlorite solution (8 μL), or 1:1 methionine solution/sodium hypochlorite solution (8 μL), were arrayed in quadruplicate in a 96-well microplate. The plate was placed in a microplate luminometer (Mithras LB-940, BERTHOLD TECHNOLOGIES U.S.A. LLC, Oak Ridge, Tenn.) at 28° C. Well by well, the choline oxidase reagent (40 μL), chemiluminescent detection reagent (40 μL) and aqueous sodium hydroxide (0.25 N, 100 μL) were sequentially dispensed and the chemiluminescent signal was recorded for 2 seconds.

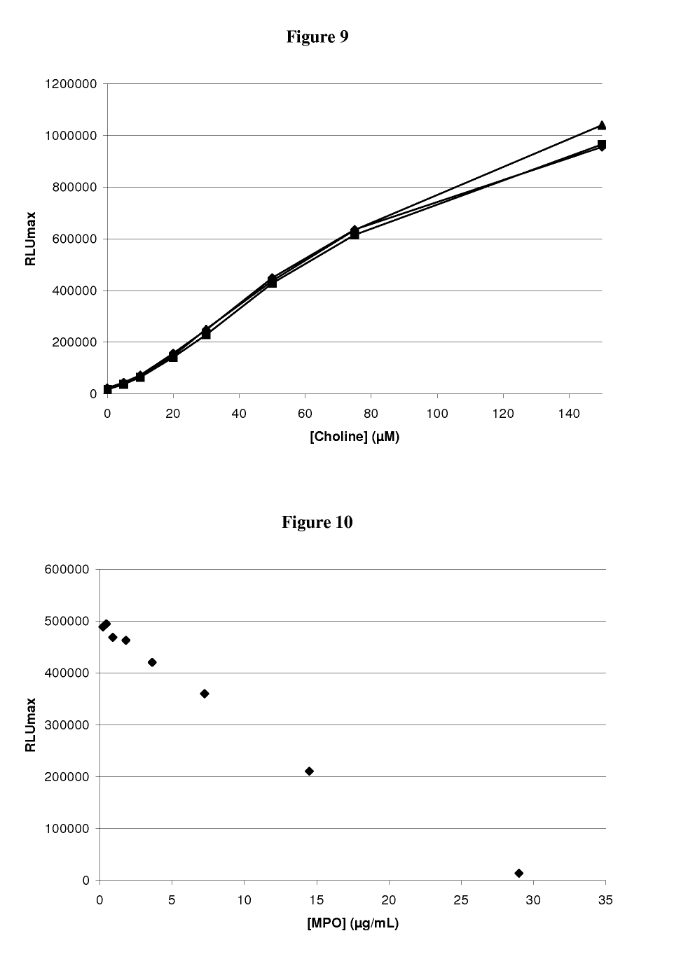

As shown in FIG. 9, neither sodium hypochlorite (100 μM) nor the further addition of methionine (1 mM) had an effect on the generation of hydrogen peroxide from choline and choline oxidase.

### EXAMPLE 3

### Dose Response for Detection of Myeloperoxidase with Hydrogen Peroxide Generated in situ with Choline/Choline Oxidase 15 Minute/28° C. Chemiluminescent Assay

### Choline Oxidase Reagent.

Choline oxidase (10 U/mL in phosphate buffer, 0.2 M, pH 8; 0.1% sodium cholate, 1 mM methionine, 100 mM sodium chloride).

### Standard Solution of Myeloperoxidase.

See preparation described in Example 1.

### Chemiluminescent Detection Reagent.

See preparation described in Example 2.

### Assay Protocol.

The choline standard solutions (4 μL) and myeloperoxidase standard solutions (4 μL), were arrayed in quadruplicate in a 96-well microplate. The plate was incubated at 28° C. for 15 minutes in a microplate luminometer (Mithras LB-940, BERTHOLD TECHNOLOGIES U.S.A. LLC, Oak Ridge, Tenn.). Well by well, the choline oxidase reagent (40 μL), chemiluminescent detection reagent (40 μL) and aqueous sodium hydroxide (0.25 N, 100 μL) were sequentially was dispensed and the chemiluminescent signal was recorded for 2 s.

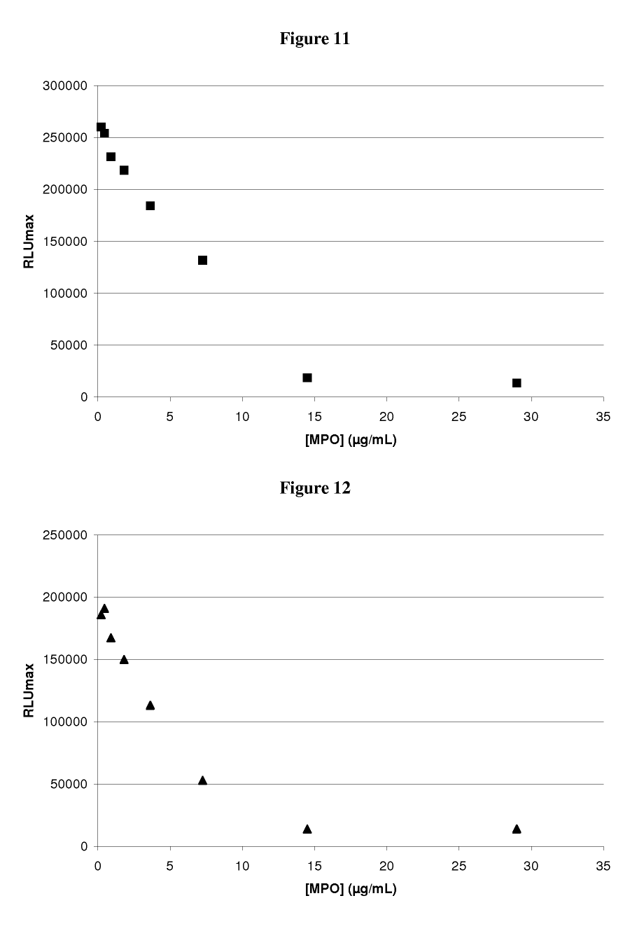

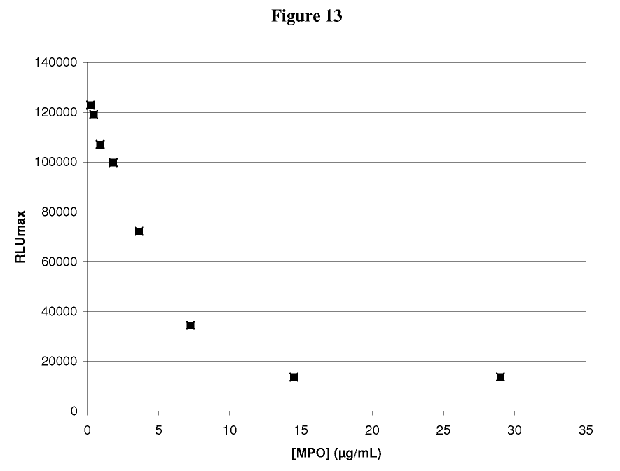

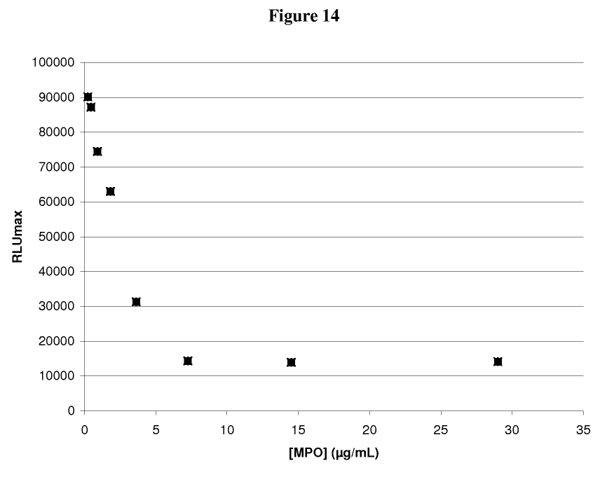

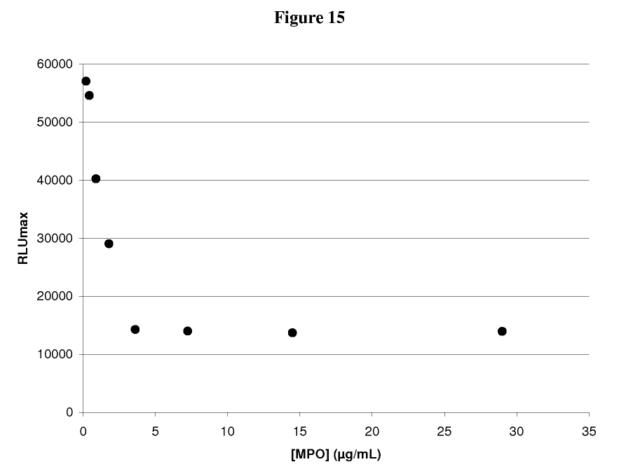

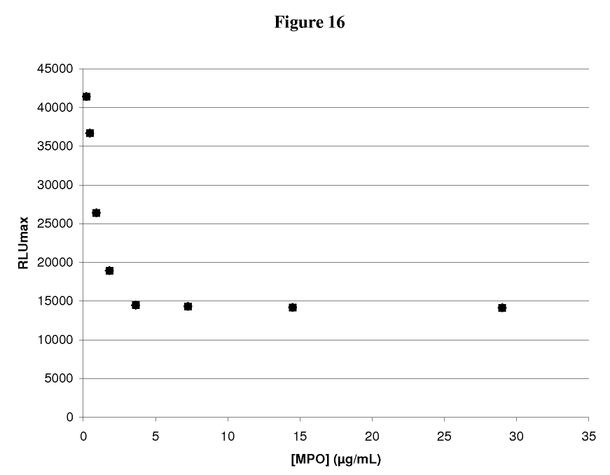

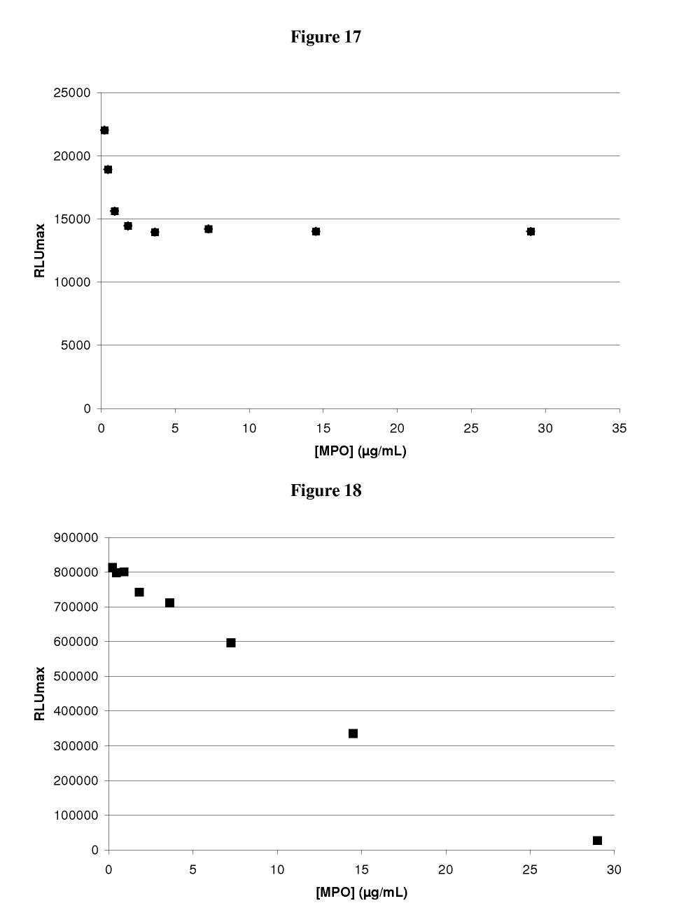

As shown in FIGS. 10-17, there was a MPO dependent dose response at all tested choline concentrations.

### EXAMPLE 4

### Dose Response for Detection of Myeloperoxidase with Hydrogen Peroxide Generated in situ with Choline/Choline Oxidase 15 Minute/37° C. Chemiluminescent Assay

The experiment of Example 3 was repeated; however, the temperature in the assay protocol was increased to 37° C.

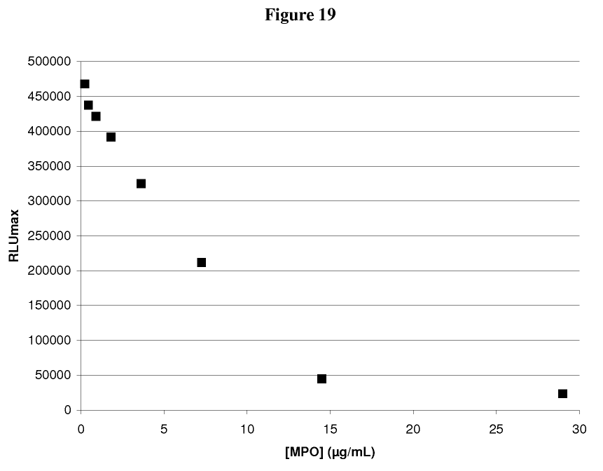

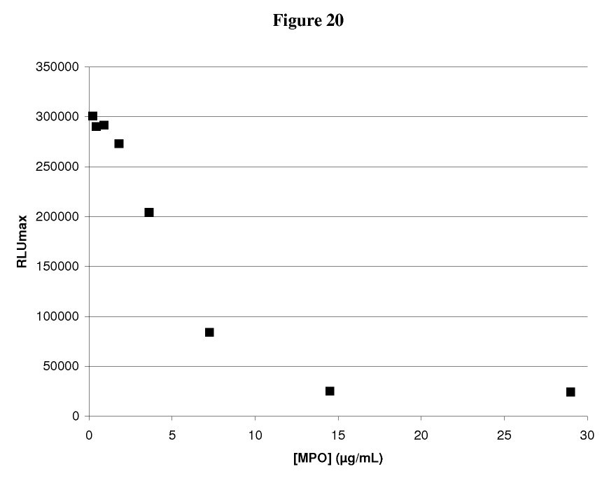

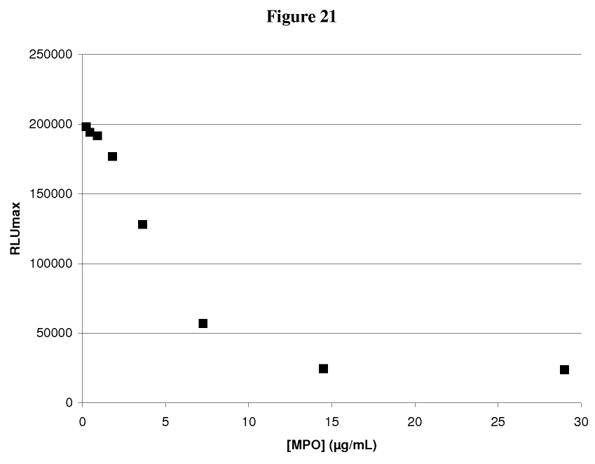

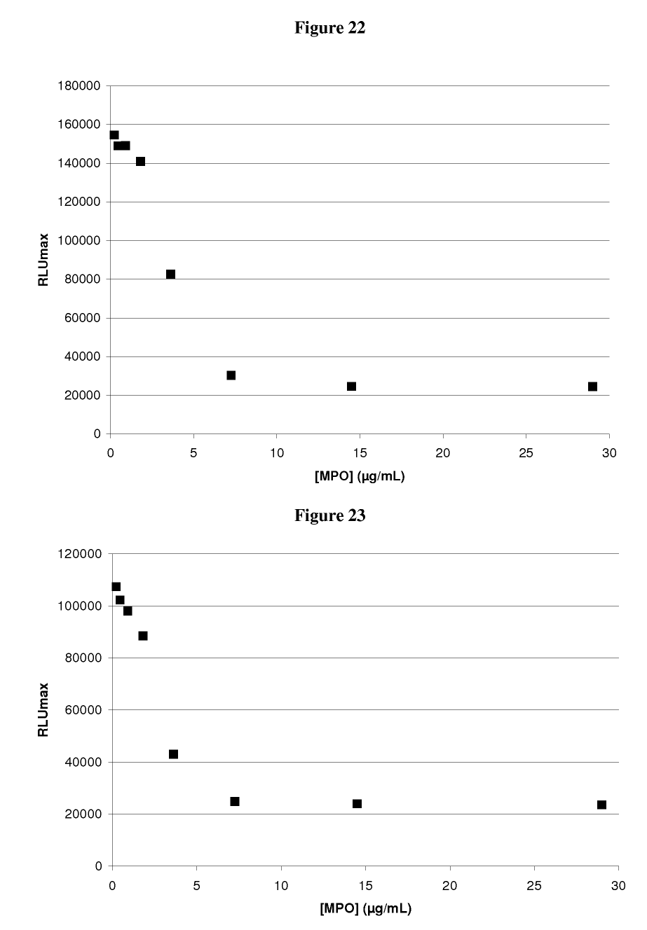

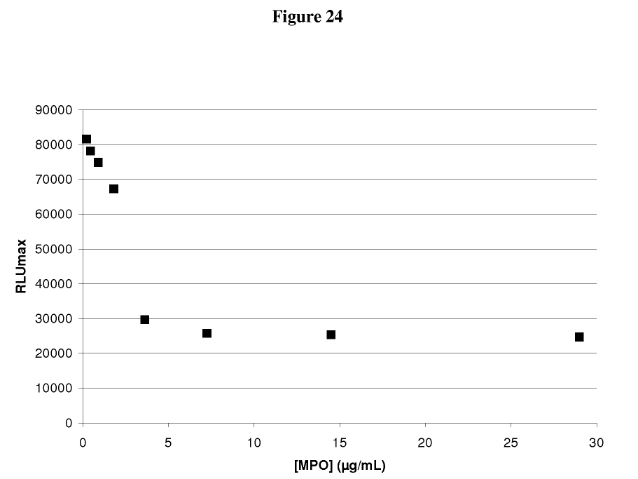

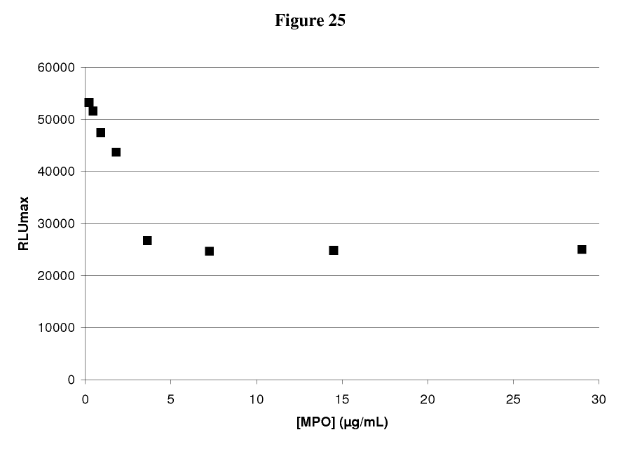

As shown in FIG. 18-25, there was a MPO-dependent dose response at all tested choline concentrations.

### EXAMPLE 5

### Dose Response for Detection of Myeloperoxidase with Hydrogen Peroxide Generated in situ with Choline/Choline Oxidase, 30 Minutes/37° C. Chemiluminescent Assay

### Standard Solution of Myeloperoxidase.

Myeloperoxidase from human leukocytes (Sigma #M6908) was diluted in phosphate buffered saline (PBS, pH 7.2) containing methionine (1 mM) to give solutions of 2900, 1450, 725, 362.50, 181.25, 90.63, 45.31, 22.66, 11.33, and 0.00 ng/mL.

### Choline Standard Solutions.

Choline standards (0, 5, 10, 20, 30, 50, 75, and 150 μM in phosphate buffer, 0.2 M, pH 8) were prepared as reported (Adamczyk M, Brashear R J, Mattingly P G, Tsatsos P H. Homogeneous chemiluminescent assays for free choline in human plasma and whole blood. Anal Chim Acta. 579(1):61-7 (2006)).

### Choline Oxidase Reagent.

Choline oxidase (1 U/mL in phosphate buffer, 0.2 M, pH 8; 0.1% sodium cholate, 1 mM methionine, 100 mM sodium chloride).

### Chemiluminescent Detection Reagent.

9-[[(3-Carboxypropyl)[(4-methylphenyl)sulfonyl]amino]-carbonyl]-10-(3-sulfopropyl)acridinium inner salt was dissolved in reagent grade water containing sodium cholate (0.1% wt/vol) to give a concentration of 250 nM.

Assay protocol. The standard choline solutions (4 μL) and the standard myeloperoxidase solutions (20 μL) were arrayed in triplicate in a 96-well microplate. The plate was incubated in a microplate luminometer (Mithras LB-940, BERTHOLD TECHNOLOGIES U.S.A. LLC, Oak Ridge, Tenn.) at 37° C. for 30 minutes. Well by well, the chemiluminescent detection reagent (40 μL) and aqueous sodium hydroxide (0.25 N, 100 μL) were sequentially added and the chemiluminescent signal was recorded for 2 seconds.

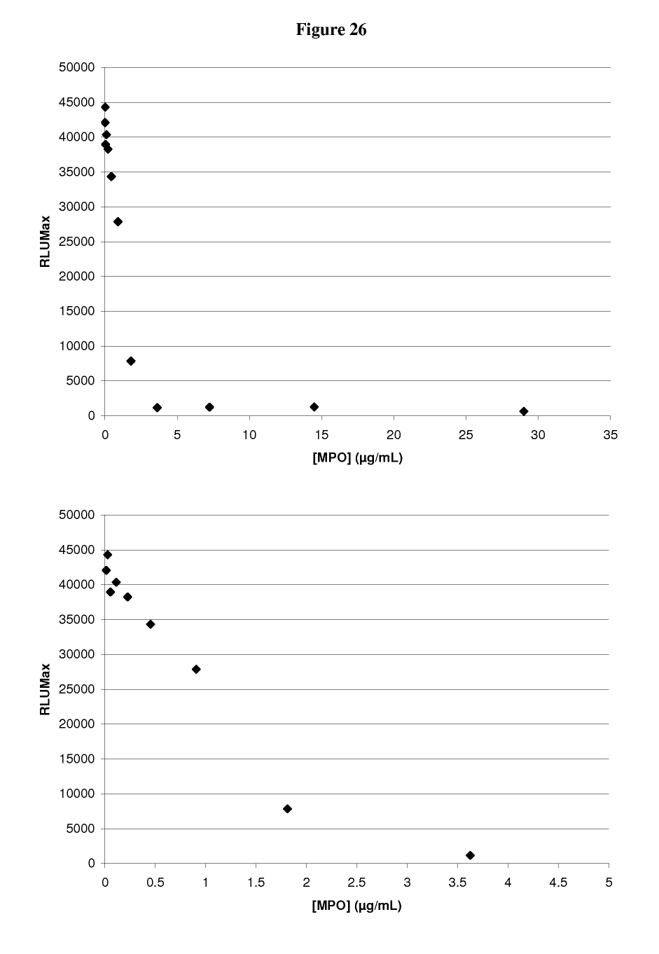

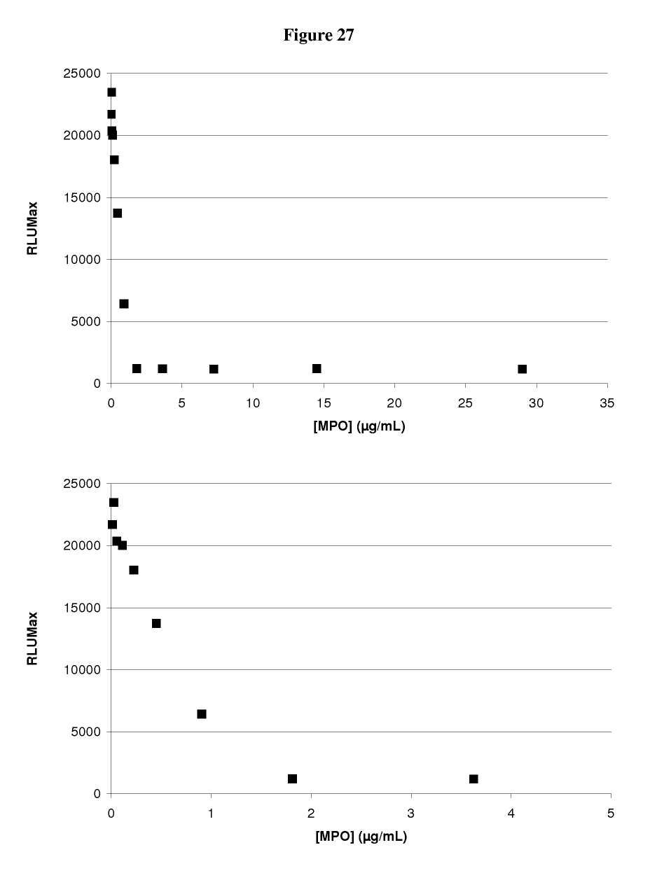

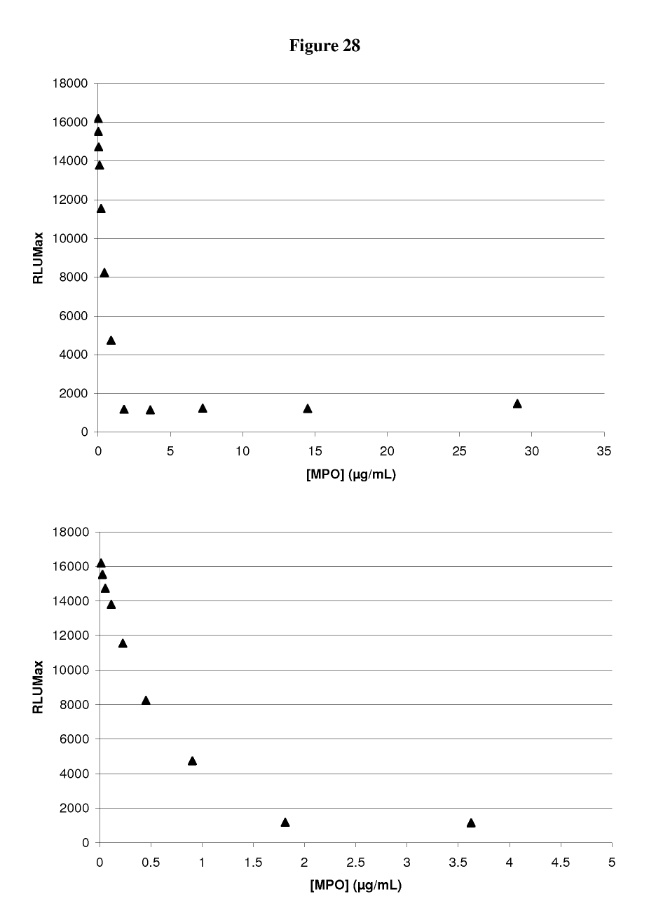

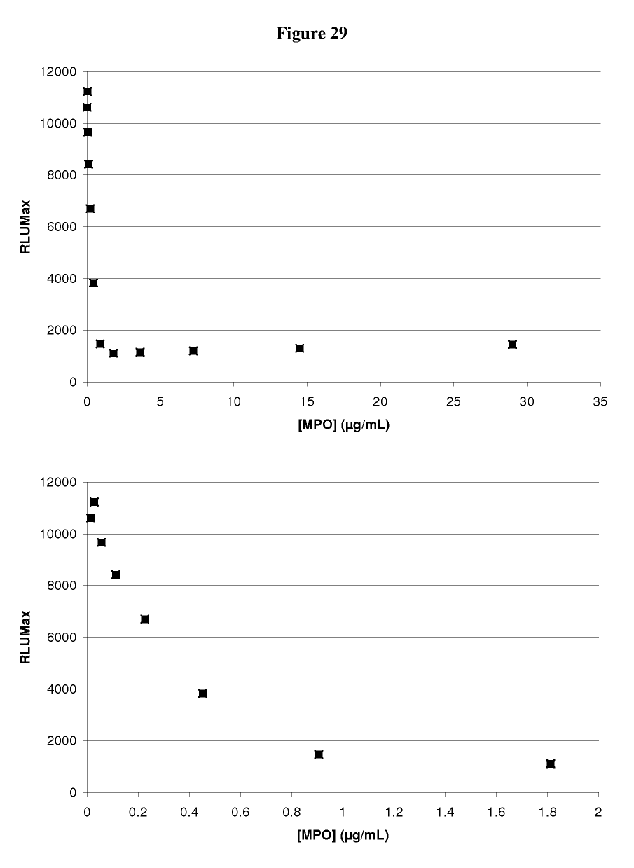

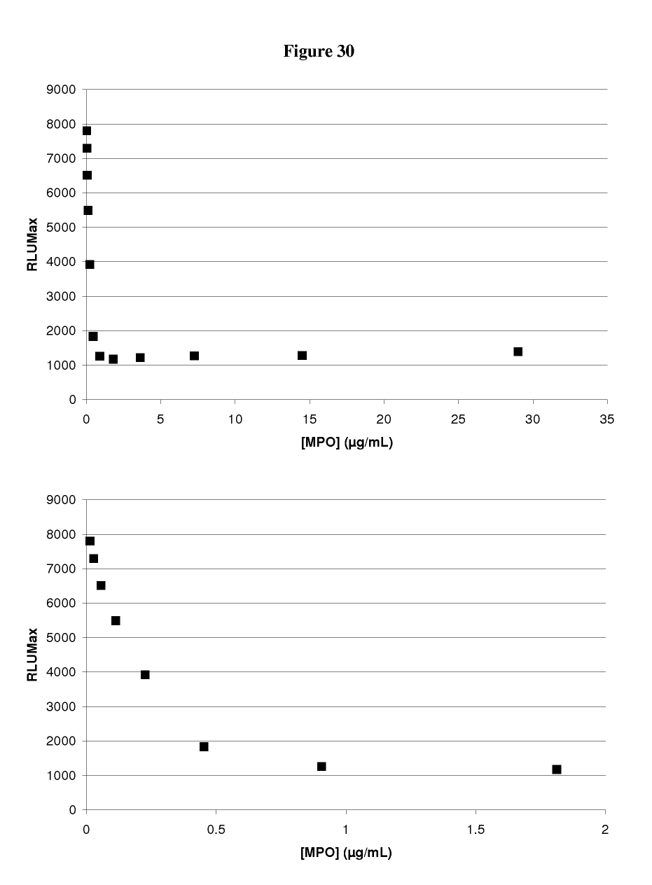

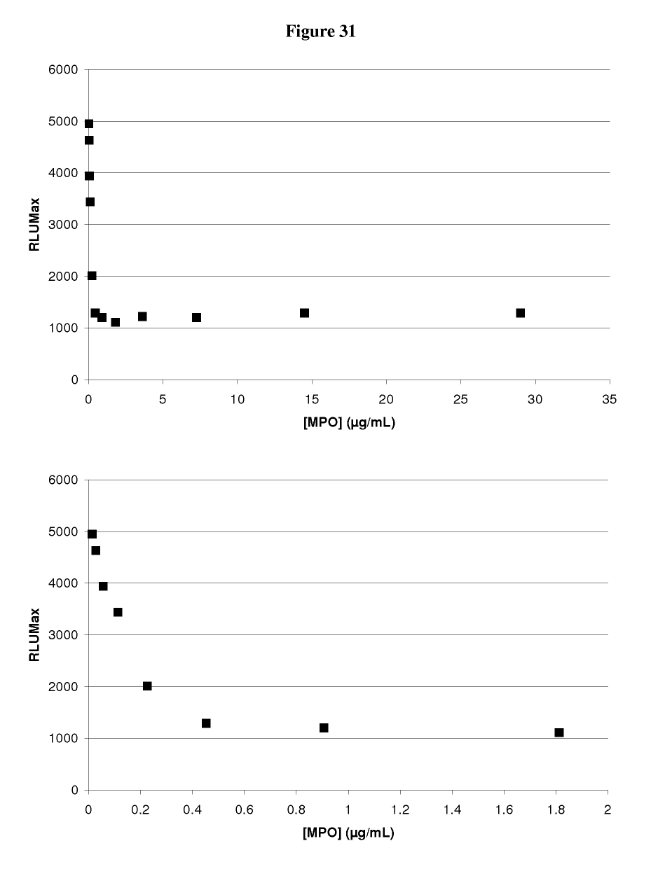

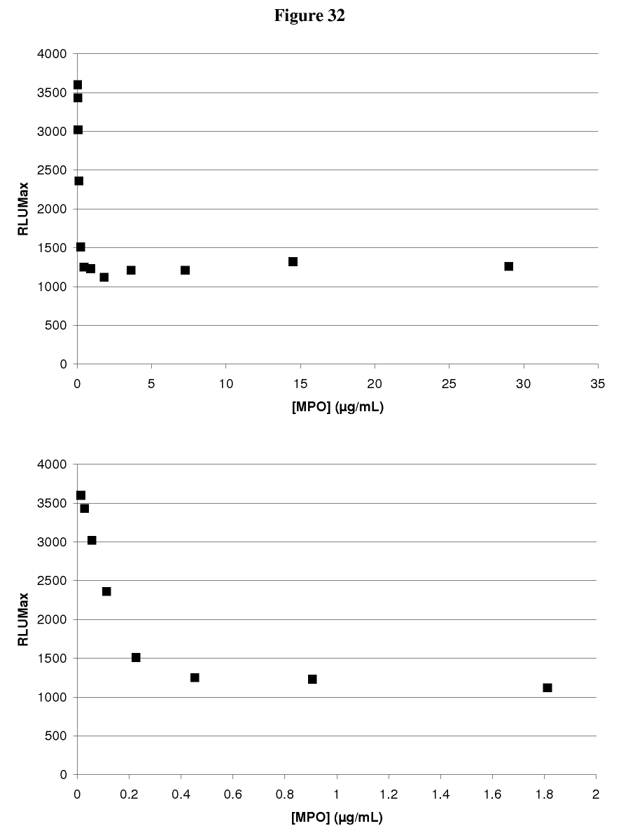

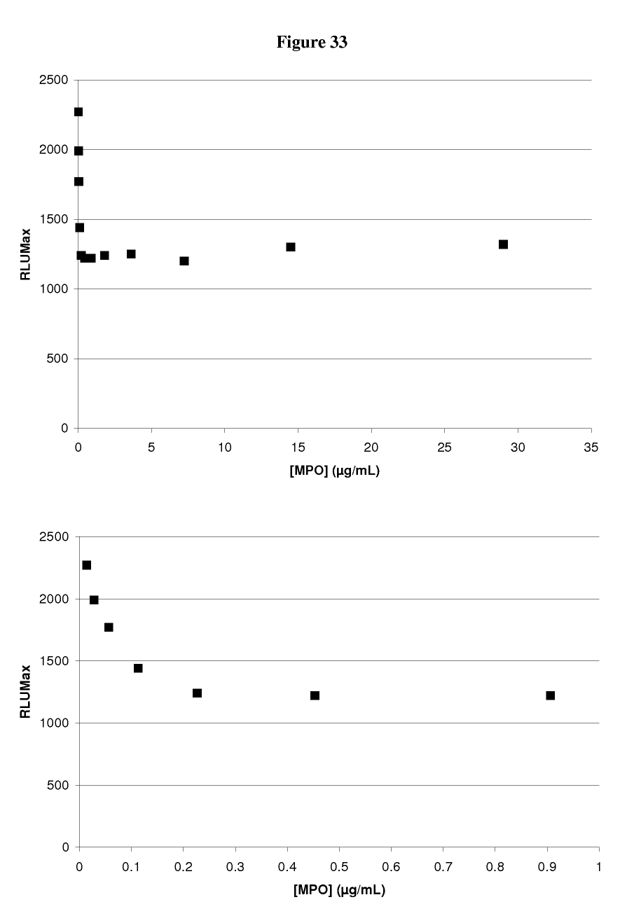

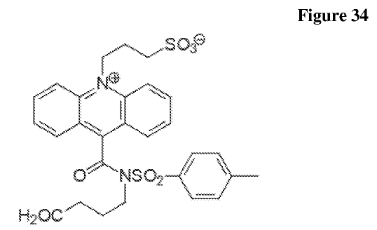

As shown in FIGS. 26-33, there was a MPO-dependent dose response at all tested choline concentrations.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of detecting haloperoxidase activity in a test sample, the method comprising the steps of:
a) adding an acridinium-9-carboxamide to a test sample;
b) generating in or providing to the test sample a source of hydrogen peroxide before or after the addition of an acridinium-9-carboxamide;
c) adding a basic solution to the test sample to generate a light signal; and
d) measuring the light generated to detect the haloperoxidase.

2. The method of claim 1, wherein the haloperoxidase is selected from the group consisting of: myeloperoxidase, thyroperoxidase, eosinoperoxidase, eosinophil peroxidase and lactoperoxidase.

3. The method of claim 1, wherein the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen.

4. The method of claim 1, wherein the hydrogen peroxide is provided by adding a buffer or a solution containing hydrogen peroxide.

5. The method of claim 1, wherein the hydrogen peroxide is generated by adding a hydrogen peroxide generating enzyme to the test sample.

6. The method of claim 1, wherein the acridinium-9-carboxamide has a structure according to formula I: 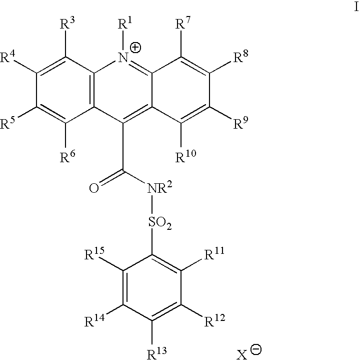
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

7. The method of claim 1 further comprising quantifying the activity of haloperoxidase in the test sample by relating the amount of light generated in the test sample by comparison to a standard curve for said haloperoxidase.

8. The method of claim 7, wherein the standard curve is generated from solutions of a haloperoxidase of a known concentration.

9. A kit for use in detecting haloperoxidase activity in a test sample, the kit comprising:
a. at least one acridinium-9-carboxamide;
b. at least one basic solution;
c. a source of hydrogen peroxide; and
d. instructions for detecting haloperoxidase activity in a test sample.

10. The kit of claim 9, wherein the acridinium-9-carboxamide has a structure according to formula I: 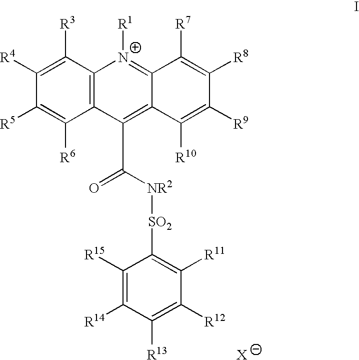
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

11. The kit of claim 9, wherein the source of hydrogen peroxide is a buffer or a solution containing hydrogen peroxide.

12. A kit for use in detecting haloperoxidase activity in a test sample, the kit comprising:
a. at least one acridinium-9-carboxamide;
b. at least one basic solution;
c. a means of generating hydrogen peroxide in situ in the test sample; and
d. instructions for detecting haloperoxidase activity in a test sample.

13. The kit of claim 12, wherein the acridinium-9-carboxamide has a structure according to formula I: 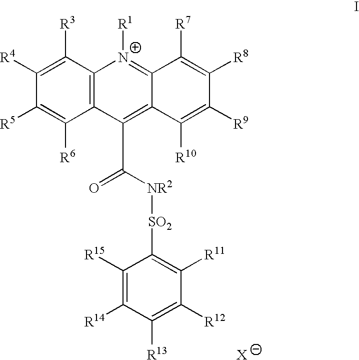
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

14. The kit of claim 12, wherein said kit further comprises instructions for generating hydrogen peroxide in situ in the test sample.

15. The kit of claim 12, wherein the means for generating hydrogen peroxide in situ is a hydrogen peroxide generating enzyme.

16. The kit of claim 15, wherein the hydrogen peroxide generating enzyme is selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate)oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.
